(19) United States
(12) Patent Application Publication
(10) Pub. No.: US 2011/0218476 A1
(43) Pub. Date: Sep. 8, 2011

(54) APPARATUS AND METHOD FOR GASTRIC BYPASS SURGERY

(76) Inventors: Stefan Josef Matthias Kraemer, Madison, NJ (US); Robert L. Wilcox, Bothell, WA (US); Thomas James Clement, Kirkland, WA (US)

(21) Appl. No.: 13/026,087

(22) Filed: Feb. 11, 2011

Related U.S. Application Data
(60) Provisional application No. 61/304,295, filed on Feb. 12, 2010.
(60) Provisional application No. 61/329,507, filed on Apr. 29, 2010.

(51) Int. Cl. A61F 2/04

(52) U.S. Cl. 604/8

(57) ABSTRACT

A medical treatment device includes an elongate member having an internal volume, a proximal end, and a distal end, the internal volume extending from the proximal end to the distal end. The medical treatment device further includes a first coupler and a second coupler, the first coupler and the second coupler coupled to the internal volume of the elongate member; a first joining member and a second joining member, the first joining member coupled to the first coupler and the second joining member coupled to the second coupler. The first joining member is configured to attach to a first biological matter location, and the second joining member is configured to attach to a second biological matter location, the second location being distal to the first location. The second coupler is configured for manipulation to align relative the first coupler such that the second biological matter location relocates adjacent the first biological matter location. The first joining member and the second joining member are configured to join the first biological matter location to the second biological matter location.

### CROSS-REFERENCE TO RELATED APPLICATIONS

This application claims the benefit of U.S. Provisional Application No. 61/304,295, which was filed on Feb. 12, 2010, and U.S. Provisional Application No. 61/329,507, which was filed on Apr. 29, 2010. The entirety of each of the priority applications is hereby incorporated by reference.

### BACKGROUND OF THE INVENTION

1. Field of the Invention

The present invention is directed to surgical methods and apparatus and more particularly to a gastric bypass procedure and apparatus to perform the same.

2. Description of the Related Art

The need for surgical procedures to address an increasing obesity problem among today's population continues to grow. A common procedure involves a gastric bypass procedure that decreases the digestive system capacity by shortening the digestive tract, in particular the small intestine. This procedure bypasses the duodenum and the upper segment of the jejunum, resulting in segregation of food (chyme) from digestive juices and enzymes. The high-glucose absorption area of the small intestine, located in the post-pyloric segments of the jejunum, is being bypassed at the same time. Existing procedures for performing such a gastric bypass procedure are either performed as open or laparoscopic procedures, and attempt to reduce the risks inherent to the procedure associated with digestive system leakage, recovery time associated with the procedure itself, and obtaining access to the digestive system. However, there is still a need for improved methods of the bypass procedure to reduce the various associated risks.

Accordingly, there is a need for an improved method and apparatus to perform a gastro-jejunal bypass to bypass the duodenum and the upper jejunum to improve recovery time and reduce risk of collateral injury.

### SUMMARY OF THE INVENTION

Methods and devices are described herein for performing bypass surgeries within the digestive tract. In one embodiment, the surgical method includes endoluminal and/or transluminal methods based on surgical principles. In one embodiment, a method of digestive tract bypass surgery is provided, comprising advancing a first device to a first target site within a digestive tract of a patient and manipulating the first device inside and/or outside the patient to move the first target site approximate to a second target site within the digestive tract. In one embodiment, the first target and the second target site are joined together to form a junction with a periphery. An opening is formed within the periphery of the junction.

In one embodiment, a method of treating diabetes is provided, comprising inserting an elongate member orally through the digestive tract, wherein the elongate member includes an internal volume, a proximal end, and a distal end, the internal volume extending from the proximal end to the distal end. A first biological matter location and a second biological matter location are located with the distal end of the elongate member. A first coupler is deployed at the first biological matter location and a second coupler is deployed at the second biological matter location from the distal end of the elongate member, the first coupler and the second coupler deploying from the internal volume of the elongate member and maintaining a coupling to the internal volume. A first protrusion coupled to the first coupler is attached to the first biological matter location and a second protrusion coupled to the second coupler is attached to the second biological matter location. The second coupler is maneuvered adjacent the first coupler by directionally maneuvering an external coupler about the second coupler. The first coupler is aligned with the second coupler, and the first biological matter location is joined to the second biological matter location by activating a first joining member coupled to the first coupler and a second joining member coupled to the second coupler. The joined portion of the first biological matter location and the second biological matter location is opened to provide for flow of bodily fluid. Disengaging and retracting the couplers from the first and second biological matter locations, and removing the elongate member from the digestive tract.

In one embodiment a medical treatment device is provided, comprising an elongate member having an internal volume, a proximal end, and a distal end, the internal volume extending from the proximal end to the distal end. The device can include a first coupler and a second coupler, the first coupler and the second coupler coupled to the internal volume of the elongate member. There is a first joining member and a second joining member, the first joining member coupled to the first coupler and the second joining member coupled to the second coupler. The first joining member is configured to attach to a first biological matter location, and the second joining member is configured to attach to a second biological matter location. The second location is distal to the first location, and the second coupler is configured for manipulation to align relative the first coupler such that the second biological matter location relocates adjacent the first biological matter location. The first joining member and the second joining member are configured to join the first biological matter location to the second biological matter location.

### BRIEF DESCRIPTION OF THE DRAWINGS

FIG. 1 is a front view schematic of the internal organs of the natural human digestive system.

FIG. 2A is a perspective view of an insertion device locating and anastomosis apparatus in accordance with one embodiment.

FIG. 2B is a cross-section view of the insertion device locating and anastomosis apparatus illustrated in FIG. 2A.

FIG. 3A is a front view schematic of the insertion device locating and anastomosis apparatus illustrated in FIG. 2A inserted orally to a target location of the jejunum.

FIG. 3B is an expanded view of the distal portion of the insertion device of FIG. 2A.

FIG. 4A is a cross-section side view of a distal end of the insertion device illustrated in FIG. 2A.

FIG. 4B is a side view of a coupler of the insertion device illustrated in FIG. 2A.

FIG. 4C is a bottom view of the coupler of FIG. 4B.

FIG. 5A is a side view of the distal end of the insertion device of FIG. 2A.

FIG. 5B is a side view of a coupler of the insertion device of FIG. 2A anchored in tissue.

FIG. 5C is a schematic view of the attached couplers at a first target and a second target of the insertion device of FIG. 2A.

FIG. 6A is a cross-section side view of a method of location adjustment of the insertion device of FIG. 2A.

FIG. 6B is a cross section of a portion of the insertion device of FIG. 2A.

FIG. 7A is a cross-section view of the couplers and indicators of the insertion device of FIG. 2A.

FIGS. 7B and 7C are cross-section views of the couplers and indicators of the insertion device of FIG. 2A shown with captive tissue located therebetween.

FIG. 8 is a schematic view of an alternative insertion device in accordance with an embodiment.

FIG. 9A is a side view of a portion of an alternative insertion device in accordance with the insertion device of FIG. 8.

FIG. 9B is a side view of a portion of an alternative insertion device of FIG. 9A in a deployed configuration.

FIGS. 10A-10B re schematic views of an alternative insertion device in accordance with an embodiment.

FIG. 11A is a side view of an alternative joining member in an undeployed configuration in accordance with an embodiment.

FIG. 11B is a top view of the joining member of FIG. 11A in a deployed configuration.

FIG. 11C is a side view of the joining member of FIG. 11B.

FIG. 12 is a side view of the joining members of FIGS. 11A-C in a joining configuration.

FIG. 13 is a schematic of a control profile of input energy for the joining member of FIGS. 11A-C.

FIG. 14A is a top view of the joining member of the insertion device of FIG. 2A.

FIG. 14B is a side view of the joining member of FIG. 14A.

FIG. 15 is a cross-section schematic of the joining of tissues by a first joining member and a second joining member of the insertion device of FIG. 2A.

FIGS. 16A-16D are cross-section schematics of an obstructing device in accordance with an embodiment.

FIG. 17 is a side view of a device to deploy a coupler shown in an undeployed configuration in accordance with an embodiment.

FIG. 18 is a side view of an LED marker device in accordance with an embodiment.

FIGS. 19A-B illustrate another embodiment of a coupler device in accordance with an embodiment.

FIG. 20 is a side view of a device to deploy a coupler shown in an undeployed configuration in accordance with an embodiment.

FIG. 21 is a side view of a device to deploy a coupler shown in an undeployed configuration in accordance with an embodiment.

FIGS. 22A-C illustrate a coupler device and a support frame in accordance with an embodiment.

FIGS. 23A-B illustrate a side view of a device to deploy a coupler shown in an undeployed and deployed configuration in accordance with an embodiment.

FIGS. 24A-B illustrate a cross-section view of a pair of deployed coupler devices in accordance with an embodiment.

FIGS. 25A-D illustrate a side view of a device and method to deploy a coupler in accordance with an embodiment.

FIGS. 26A-B illustrate a side view of a delivery device to deploy a coupler shown in an undeployed and undeployed configuration in accordance with an embodiment.

FIGS. 27A-B illustrate a side view of a portion of a delivery device to deploy a coupler shown in an undeployed and deployed configuration in accordance with an embodiment.

FIGS. 28A-C illustrate a side view of a portion of a delivery device to deploy a coupler shown in an undeployed and deployed configuration in accordance with an embodiment.

FIGS. 29A-C illustrate a side view of a portion of a delivery device to deploy a coupler shown in an undeployed and deployed configuration in accordance with an embodiment.

FIGS. 30A-B illustrate a side view of a portion of a delivery device to deploy a coupler shown in a partially deployed and deployed configuration in accordance with an embodiment.

FIG. 31 is a side view of a valvular device configuration formed in accordance with an embodiment.

FIGS. 32A-B illustrate a top view of tissue forming a valvular device in accordance with an embodiment.

FIG. 33 is a side view of a valvular device configuration formed in accordance with an embodiment.

FIGS. 34A-B illustrate a top view and side view of tissue forming a valvular device in accordance with an embodiment.

FIGS. 35A-B illustrate a coupler device and method in accordance with an embodiment.

FIGS. 36A-B illustrate a coupler device in accordance with an embodiment.

FIG. 37 is a top view of a welded tissue region formed in accordance with an embodiment.

FIGS. 38A-B illustrate a coupling device and method in accordance with an embodiment.

FIG. 39 is a perspective view of a coupler device in accordance with an embodiment.

FIG. 40 illustrates a coupling device and method in accordance with an embodiment.

FIG. 41 is a side view of a coupler device in accordance with an embodiment.

FIG. 42 is a side view of a portion of a coupler device in accordance with an embodiment.

FIG. 43 is a side view of a coupler device in accordance with an embodiment.

FIG. 44 is a side view of a valvular device in accordance with an embodiment.

### DETAILED DESCRIPTION OF THE PREFERRED EMBODIMENTS

In the following description of the preferred embodiments, reference is made to the accompanying drawings which form a part thereof, and in which is shown by way of illustration a specific embodiment. It is understood that other embodiments may be utilized and structural changes may be made without departing from the scope of the present invention.

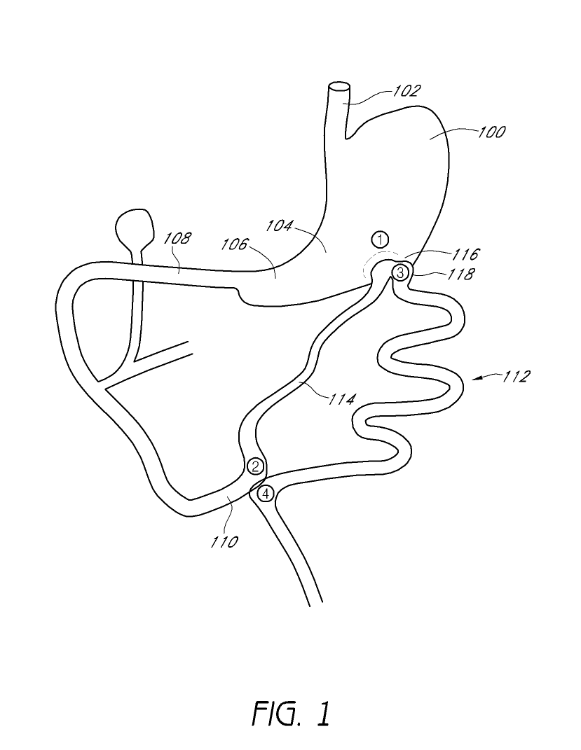

FIG. 1 illustrates the human digestive system with a first target 116 and a second target 118 identified for the locations within the digestive system that will be anastomosed to provide for a bypass of the duodenum and subsequent portions of the digestive tract. The digestive tract shows the esophagus 102 entering into the stomach 100 pouch. The stomach leads into the fundus 104, the corpus, and the pylorus 106, which subsequently transitions to the small intestine, that comprise the duodenum 108, the jejunum 110, and the ileum.

FIG. 1 illustrates a gastro-jejunal bypass procedure to bypass the duodenum and the upper segment of the jejunum. In one embodiment, this bypass procedure can be used to treat and/or reverse type 2 diabetes mellitus (T2DM) and/or treat other conditions. FIG. 1 illustrates that the first target 116 and the second target 118 have been joined, or anastomosed at a first location 120, and a third 122 and a fourth target 124 have been joined, or anastomosed at a second location 130. Various methods and apparatus for marking, moving and joining these targets 116, 118, 122, 124 according to certain embodiments will be described in further detail below. As illustrated in FIG. 1, the joining of the first target 116 and the second target 118 creates a bypassed region, or anisoperistaltic loop 114, downstream of the pylorus and upstream of the joint occurring between the first and second targets 116, 118. The first target is generally located in the portion of the intestine approximately 60 cm downstream from the pylorus, however, the location can range from 30 cm to 120 cm. The downstream portion of the jejunum, or isoperistaltic portion, becomes the alimentary loop 112. In modified embodiments, the first and second targets can be located at different positions and/or additional targets can be identified and used. In another embodiment, the anisoperistaltic loop can be lined with a liner to allow for food or chyme passage as well in addition to the previously described alimentary loop. In yet another embodiment, either of the two loops may be narrowed down using some kind of application of energy to the intestinal wall with the goal of shrinking its lumen, making it less amenable for the transportation of food or chyme. In another embodiment, either one of the two lumina can be fitted with a one way valve by means of tissue reconfiguration, which prevents chyme from passing in one of the two lumina.

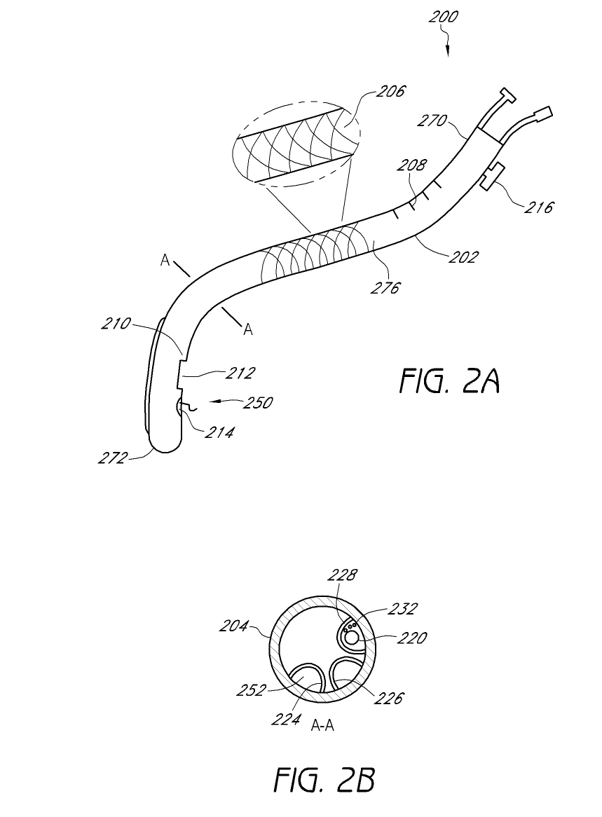

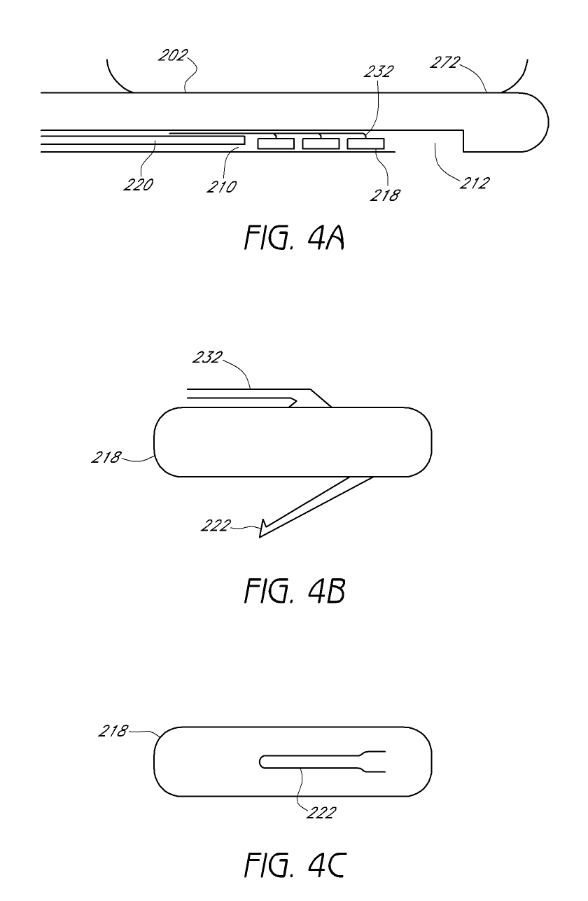

FIGS. 2-7 illustrate one embodiment of the system having an insertion device 200 that can be delivered transorally to a patient to perform the gastro-jejunal bypass procedure endoluminally, or from within the hollow organ. In the illustrated embodiment, the insertion device 200, otherwise referred to as a catheter, can include a shaft-like tube that further includes supports 206. The catheter 200 has a proximal end 270 and a distal end 272. In one embodiment, the supports 206 include two counter-helical coils that provide strength to the structural body of the catheter 200. The structural strength advantageously provides for the capability to house, transport, and control a viewing mechanism 250, an inflation member 240, and couplers 218, all of which can be utilized by the system to perform the bypass procedure. The catheter can include separate internal lumens 224, 226, 228 that individually house the described features, the camera, the inflation gas, and the magnet driveshaft and electrical connections, respectively, for at least a portion of the catheter length. The internal lumens 224, 226, 228 can be attached to the inner diameter surface of the catheter 200. In another embodiment, the internal lumens 224, 226, 228 can be freely disposed in the catheter 200.

The catheter 200 can include length markings 274 along the length of the lubricious outer diameter surface 276 that can provide an indication of how far the catheter has travelled during insertion into the patient. The catheter 200 system can further include an inflation member 240, or a balloon, that is coupled to an upstream inflation lumen 242. The balloon 240 can be coupled to the external surface of the catheter 200 distal end 272. Inflation lumen 242 provides a conduit for a gaseous or liquid pressure source to inflate/deflate the balloon 240. The gaseous source can be any suitable medical grade gas, e.g. helium, carbon dioxide, ambient air, liquid sterile water, saline, gels, or the like.

The viewing mechanism 250, or camera, can be disposed within the catheter 200. The camera 250 can provide a side-looking view via the application of a 90° optic tip that is directed through a second aperture 214, or camera window. In other embodiments, the camera 250 can view in the distal direction via a 180° optic tip. The camera 250, its viewing tip, and the camera window 214 are located within the catheter 200 adjacent the distal end 272. The camera 250 can be coupled to a viewing connector 252, or camera cable, that extends proximally to the controller 216. The orientation of the camera 250 can be controlled by the controller 216 via the camera cable 252. In one embodiment, the camera 250 includes an articulating head that can be controlled by the controller 216. In another embodiment, the camera 250 can be controlled by an external control mechanism not associated with the controller 216.

In the illustrated embodiment, the system further includes mechanisms to attach, locate, and/or mark the target spots. In the illustrated embodiment, the system can include three couplers 218, or magnets. In some embodiments, the system can include 2, 4, 5, or more magnets 218. The magnets 218 can include protrusions 222, or hooks, or other attachment mechanism that provide a positive attachment function to the tissue at the desired target locations. In this arrangement, the hook 222 can include a free end and a coupled end, where the free end generally urges away from the magnet 218 bottom surface, or outer diameter directed surface, in a spring-like manner. In other embodiments, the magnets can be glued to the surface using tissue glues. In other embodiments, magnets can be attached to the internal organ surface with the help of a specialized surface, e.g. Velcro or the like. In other embodiments, the magnets 218, or markers, can be clipped to the tissue surface. The free end of the hook 222 is captively retained against the catheter 202 or internal lumen 224 wall. The magnets 218 are located adjacent the distal end 272 and are controlled by connector 220, or the magnet driveshaft. The magnets 218 can be coupled to power members 232, which provide an electrical connection for a joining member 230, otherwise referred to as an electrode. As will be explained below, the electrodes 230 can be utilized to perform the anastomosis via tissue welding of the tissue walls for the two target locations to be anastomosed. The magnets 218 can be temporarily stored in the deployment compartment 210 located adjacent the distal end 272. The magnets 218 can be deployed from the catheter 200 distal end 272 through a first aperture 212, or deployment window.

The magnets 218, driveshaft 220, balloon 240, camera 250, and catheter 200 are directed, and can be administered via controller 216 located at the catheter 200 proximal end 270. In one embodiment, the magnets 218 can include an indicator 260, or sensor, that provides a signal and feedback to a controller module to determine distance and intermediate obstacles between an adjacently located indicator 260 on a separate magnet 218. Alternatively, the sensor 260 is only located on one of two adjacently located magnets 218.

In the illustrated arrangement, the magnet 218 can include a flat oblong geometry with the hook 222 being steel-spring fish-hook type protrusion coupled to the outer diameter adjacent surface of the magnet 218. The hook 222 can be coupled at an angle and restrained with a pre-load adjacent the magnet 218 outer diameter surface. The three magnets 218 can be stored in a single-file series fashion within the catheter storage compartment.

It should be appreciated while magnets 218, driveshaft 220, balloon 240, camera 250, and other components are illustrated as part of a single catheter 200, in modified embodiments, these components can be rearranged and positioned into separate components or catheters.

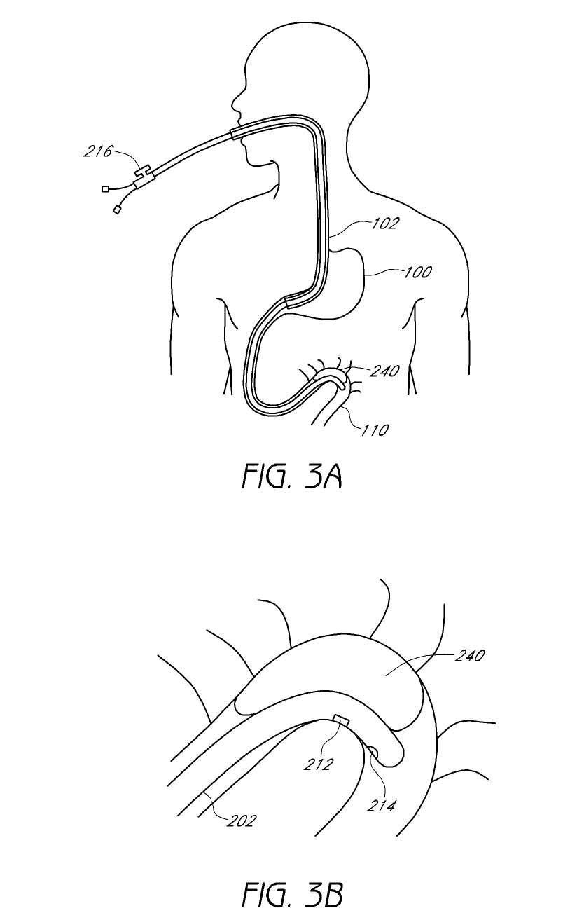

A method of performing the gastro-jejunal bypass according to one embodiment will now be explained in detail. In one arrangement, the procedure is performed transorally (see e.g., FIG. 3A) in order to reduce the risk of complications from surgical intervention and reduce the recovery time of the patient. The procedure can be performed using the catheter 200 described above or with a modified system configured to perform the methods and steps described below. As will be described below, in one arrangement one or more target sites are marked, the target sites are approximated (i.e., brought in close spatial relationship to each other), and then the tissues are anastomosed. In certain embodiments, a second anastomosis can be performed, obstructions can be placed in the bypassed tissue lumens, the anastomosis can be protected intraluminally, and/or the security of the anastomoses can be tested.

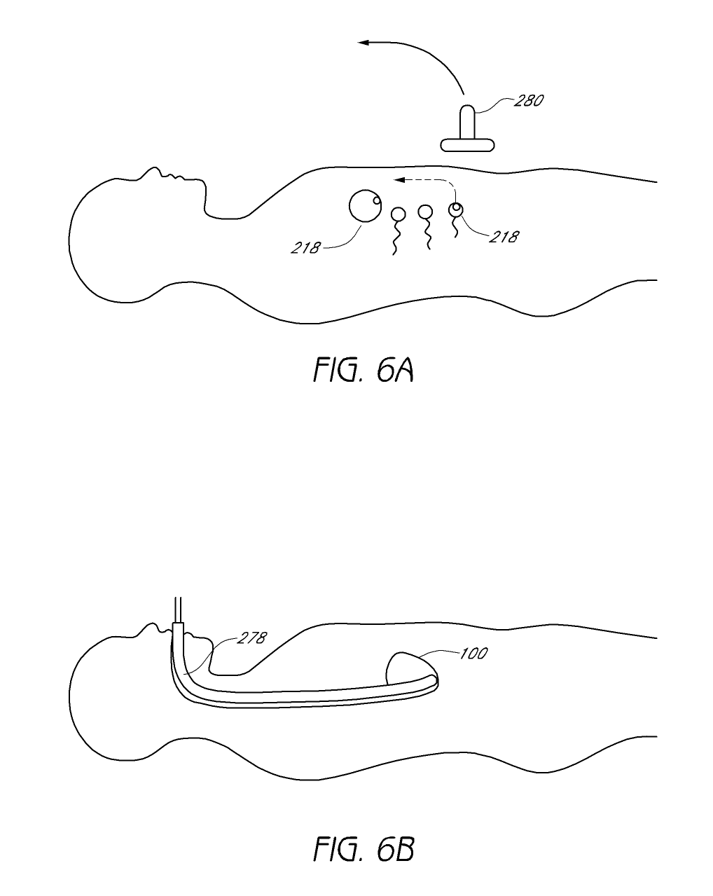

In one embodiment, the catheter system 200 is deployed through a guide catheter 278 (FIG. 6B). The catheter 200 deploys and attaches the magnets 218, maneuvers the second target 118 adjacent the first target 116, anastomoses the adjacent first and second targets 116, 118, slits the tissue encompassed by the anastomosis periphery, and blocks the pylorus 106 exit from the stomach 100. The catheter 200 system distal end 272 can be inserted through the mouth and through the esophagus, stomach, and then the pylorus with a simple guide catheter. Natural motility of the intestine in combination with the motive force of operator pushing the driveshaft 220 via controller 216 can assist in delivery of the catheter distal end 272 tip at the second target 118.

The distal progress of the catheter 200 distal tip can be monitored by observing the length markings, by diaphanoscopy, by fluoroscope or ultrasound imaging, or the like. The catheter shaft can be rotated by the controller 216 such that the camera 250 can observe the tissue wall to aid in determining the vascularity of said adjacent tissue wall. The magnet 218 can be deployed in to the tissue wall with the least vascularity by directing the camera at the optimal wall location and fully inflating the balloon 240, which can be mounted 180° opposite the deployment compartment 210. In other embodiments, the magnet 218 can be deployed into the tissue wall at any distal length location, e.g. based on a proportion of overall digestive tract length, or the like.

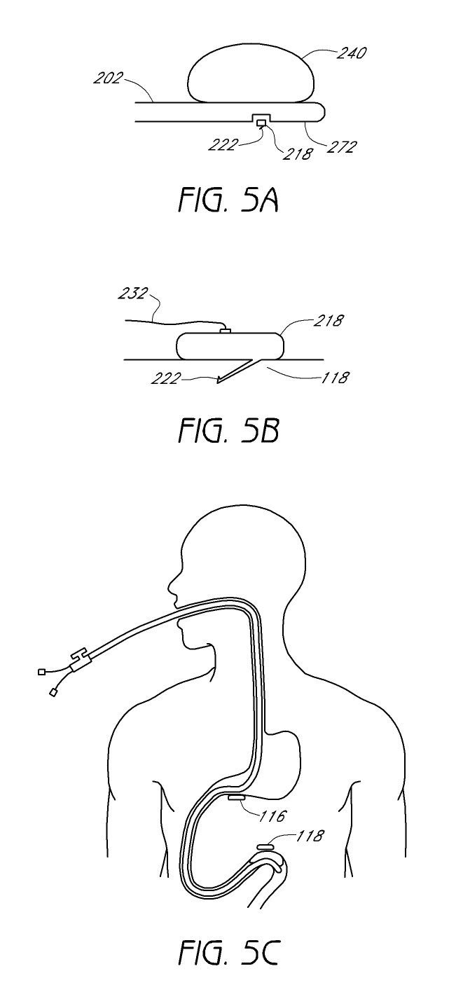

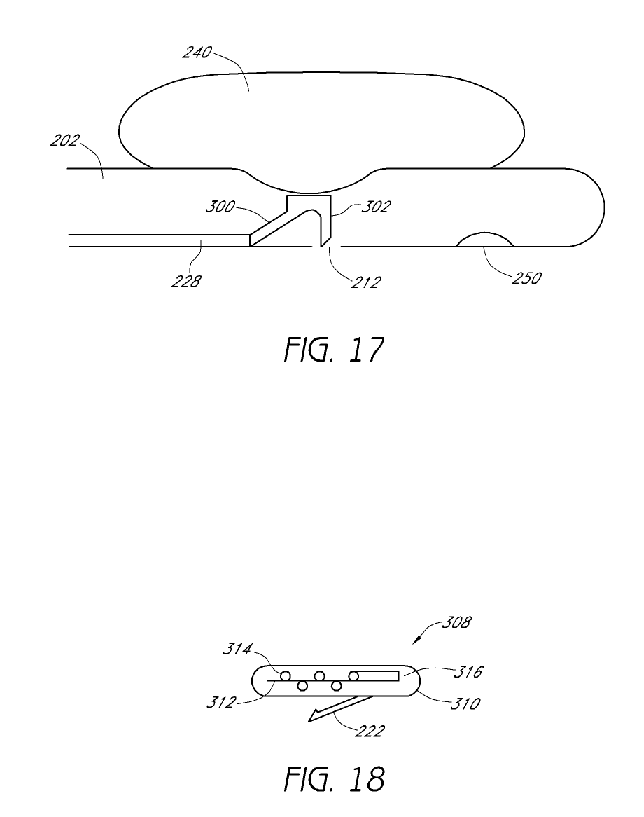

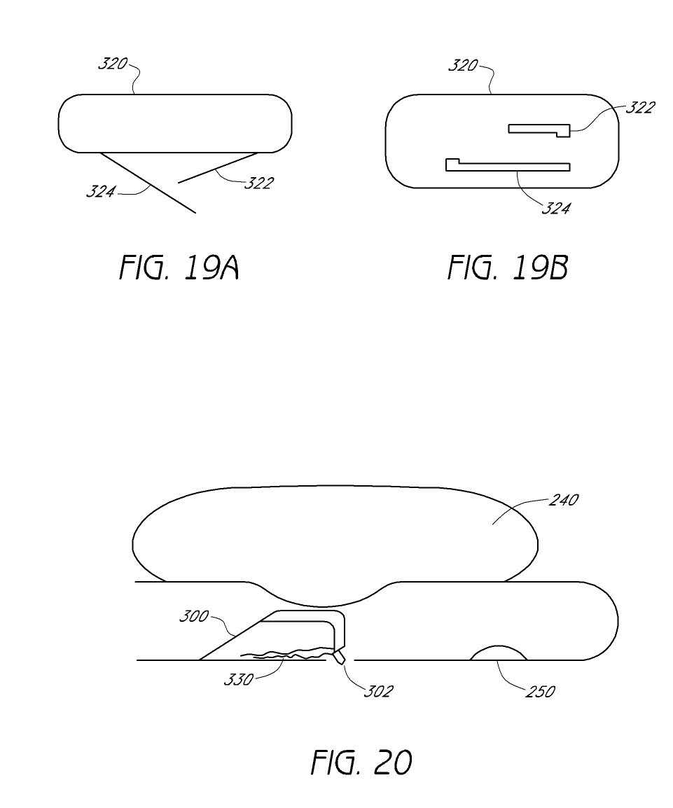

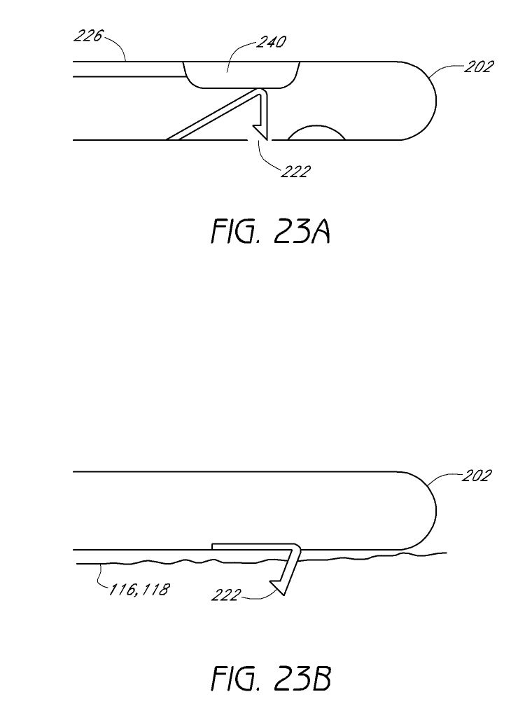

In the illustrated embodiment, the operator can deploy the magnet 218 by pushing the magnet 218 distally by exerting a distally directed force on the driveshaft 220, thereby placing the magnet 218 into the deployment compartment. As the magnet exits the catheter 200 deployment window 212, the hook 220 springs in a radially outward direction and engages the tissue wall. The catheter 202 can then be withdrawn to anchor the magnet into the tissue wall. The balloon can be deflated and the catheter is disengaged from the magnet. A cable for each of the three magnets can extend from the magnet back into the catheter 200 and to the controller 216. In other embodiments, different methods of attaching the magnet 218 to the tissue wall can be accomplished, e.g. a balloon attached to a hinged needle (see FIG. 17), multiple spring pins located distally and proximally on the magnet 218 extending longitudinally at opposing angles to each other (see FIGS. 19A-19B), or the like. In other embodiments, the magnets 218 can be permanently or semi-permanently attached to the catheter 202 (see FIGS. 23A-B), and the catheter would be attached to the tissue wall with the magnet 218. The placement of a second magnet 218 adjacent first target 116 can be accomplished by repeating the above process, such that a magnet 218 is attached, or anchored, to the tissue at both the first and second targets 116, 118 (see FIG. 5C).

The embodiment depicted in FIG. 17 shows the catheter 202 with an injection lumen 228 connected at an angle to the spring 300 retracted injection needle 302 in at an at-rest, or unloaded, condition. The needle 302 can be deployed through the deployment window 212 by the inflated balloon 240 into the tissue. The balloon 240 can also push a hinge to drive the needle 302 out of its lumen/housing 228 and into the target. A volume of dye can be injected by a syringe attached to the lumen 228. Dye can be visible to an observer inside and outside the lumen 228, for example by laparoscope or open surgical access.

In other embodiments, the marker can be a component of something other than an electromagnet. For example, in one embodiment, a metallic or radiopaque polymeric object can be visually detected or detected by external imaging devices. Another marker embodiment can include an LED marker 308 (see FIG. 18) with batteries 316 to provide a light-emitting beacon. The LED device 308 can include a circuit board 312 and LED's 314 powered by batteries 316 and visible through a clear housing 310 encasing and forming the LED device 308. In other embodiments, the marker can comprise an RFID tag (not shown). In yet another embodiment, a marker can comprise a piezo device (not shown) to deliver a sound beacon. In the embodiments described above, the magnet can comprise a constant magnet, rather than an electromagnet. The dye can also comprise a liquid containing magnetic filings.

A coupler 320 can include a multiple spring pin embodiment as depicted in FIGS. 19A-B that can include two opposed spring-steel pins 322, 324 that can deploy at angles of about 30 to 45 degrees, although other angles are possible, with one pin longer than the other and at a greater pitch. The pins 322, 324 can be set by first drawing the catheter back to set the long pin 324, and then pushing forward to set the shorter pin 322. This apparatus of attachment can be readily extracted from the wall without tissue tearing.

In some embodiments, as depicted in FIG. 20, a suture 330 can be deployed by a puncture into the wall, leaving two exposed tails as, for example, a method of marking the location. This can be done with a hinge mechanism and cutting needle 302 driven by the balloon 240 inflation, as described above. In some embodiments, the catheter can use welding as a marking device. In other embodiments, an electrical resistance heater, an arc generator, a fiberoptic for delivering laser energy, or a lumen for delivering chemical etchant can leave scarred or even charred tissue behind as a mark.

In other embodiments, as depicted in FIG. 21, the marker 218 can be discharged from a discharge cylinder 340 at high velocity into the wall of the lumen, rather than applying force with a balloon. For example, an inelastic, flexible lumen 342 such as stainless steel or polyimide or PEEK, can deliver high pressure air to a projectile such as a miniature harpoon type needle 344 configuration. The harpoon 344 can be attached, or tethered, to the marker 218 by a suture. The harpoon 344 can puncture into the tissue due to the immense pressure developed at its tip, but would not appreciably displace tissue due to the minimal momentum generated. In other embodiments, conventional, FDA-cleared clips, metal or plastic, radio opaque or not, or the like, can be deployed as tissue markers 218.

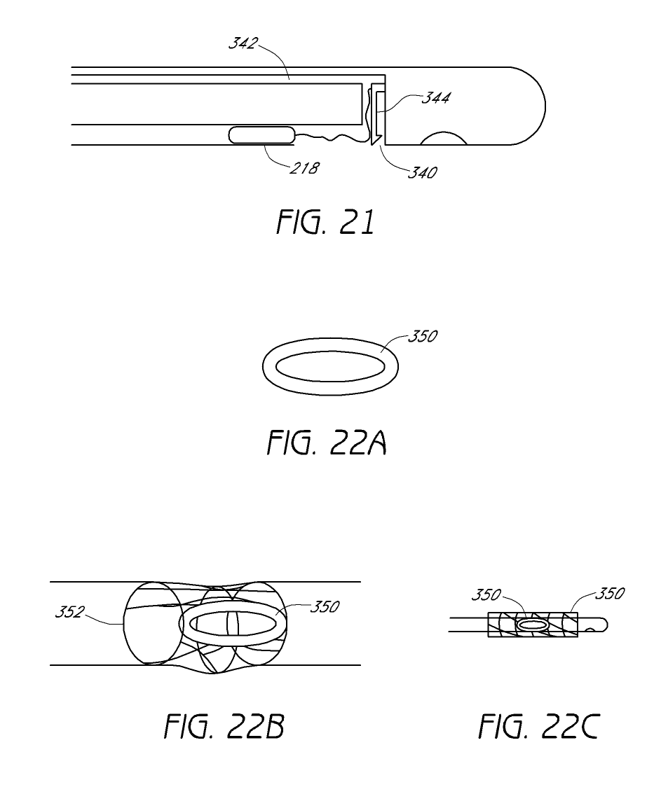

In other embodiments, as depicted in FIG. 22, the marker 350 can have a particular shape that can be deployed as a combination of marker and engineered anastomosis design orifice template 350. The shape of an optimized anastomotic orifice 350 can be oblong with the long axis along the axis of the lumen for optimal welding. The length and end radii of the oblong shape 350 can further be optimized to prevent tearing and to ensure good chyme movement, such that the oblong shape can become circular and near to the original lumen in diameter, under the influence of the hoop tension in the lumen after the anastomotic opening is formed. The shaped marker 350 can also have walls designed to ensure leak-proof joints during the welding process. The shaped marker 350 can include or be a stent 352 externally mounted on the catheter to be deployed by the balloon 240. A stent 352 can maintain the lumen opening and prevent inadvertent contact with unintended walls during the welding process. The stent 352 can also have the benefit of advantageously creating a barrier to prevent leakage from the lumen to the abdominal cavity. The material can be a long-term implant such as steel or nitinol, plastics such as PTFE, or carbon fiber, or a bioresorbable material, or can be removed after the procedure. The material can also be any combination of the above, e.g. with absorbable and non-absorbable components, of which the non-absorbable components remain in place for a certain period of time or for the long-term.

A method of approximating the two targets 116, 118 according to one embodiment is illustrated in FIG. 6. In the illustrated embodiment, the distally attached magnet 218 at the second target 118 is manipulated to bring the second target 118 proximally adjacent the first target 116. The two targets 116, 118 can be brought together for anastomosis by a combination of insufflation, and magnetic manipulation. For example, in one embodiment, with the patient in a supine position, the balloon 240 on the catheter can be inflated with the inflation gas, e.g. helium, carbon dioxide, or the like. The balloon inflation can lift the target intestine loop anteriorly. An external, hand-held magnet 280 can be used to attract the distal magnet 218 and the target anisoperistaltic loop 114 further anteriorly and cephalad to the stomach 100.

The stomach 100 can optionally (or in addition) be moved caudally with a pushing element 290, not shown, mounted slidably on the catheter. The two magnets 218 can be brought into proximity to each other with the external magnet 280. The electromagnets can be switched on and off to prevent undesired movement, or to select which of the anchored magnets 218 to move, during the manipulation procedure. Manual manipulation through the abdominal wall, position changes (e.g. Trendelenburg and anti-Trendelenburg) and shaking of the patient, and other gross physical manipulation can be used to assist in mobilizing and approximating the target organs. The force required to move the bowel, or second target 118, is minimal, within the range of 0-20, and more particularly 0-10, pounds force.

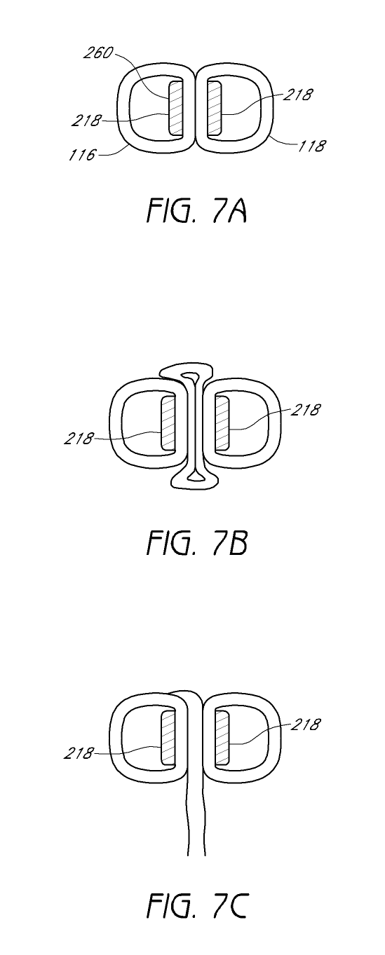

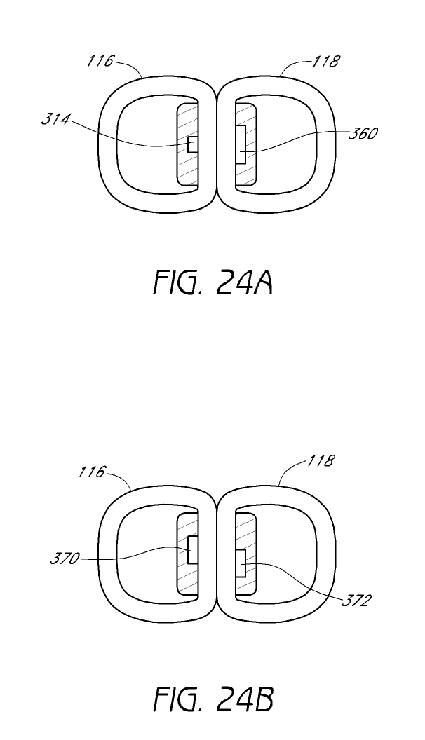

In the illustrated embodiment of FIGS. 7A-7C, the indicators 260, or Hall effect sensors, can be utilized after the target tissues at locations 116, 118 are generally approximated. The sensors 260 can be coupled to, or adjacent to, the magnets 218, and operate to measure the distance between the anastomosis sites. The distance measurement can be used by the operator to ensure or check that no additional tissue, e.g. an unintended, deflated loop of intestine, is captured between the approximators, or magnets 218 (see FIG. 7B). The joining sites, or first and second targets 116, 118, can be disengaged, moved, and reengaged until correctly positioned opposite each other if the sensors indicate extra tissue is captured. In other embodiments, a light indicator, or photo detector 360, that evaluates intensity or color of, for example, an LED 314, when shone through the anastomosis site, rather than a Hall effect sensor configuration, can be used to indicate distance or captive tissue (see FIG. 24A). In other embodiments, an acoustic sensor configuration, such as a PZT source 370 and a PZT microphone 372, can be used to indicate distance or captive tissue (see FIG. 24B). In still other embodiments, a simple electrical impedance measurement can indicate tissue wall thickness, and thus reveal the existence of extra tissue. If omentum is captured between the two target tissues the omentum can be left in place and welded through (see FIG. 7C). Welding through the omentum leaves a perforation in the omentum that will tend to close and heal around the anastomosis, advantageously improving the leak-proof characteristics of the tissue joining joint. After the two magnets 218 are sufficiently located the target areas are ready for tissue welding.

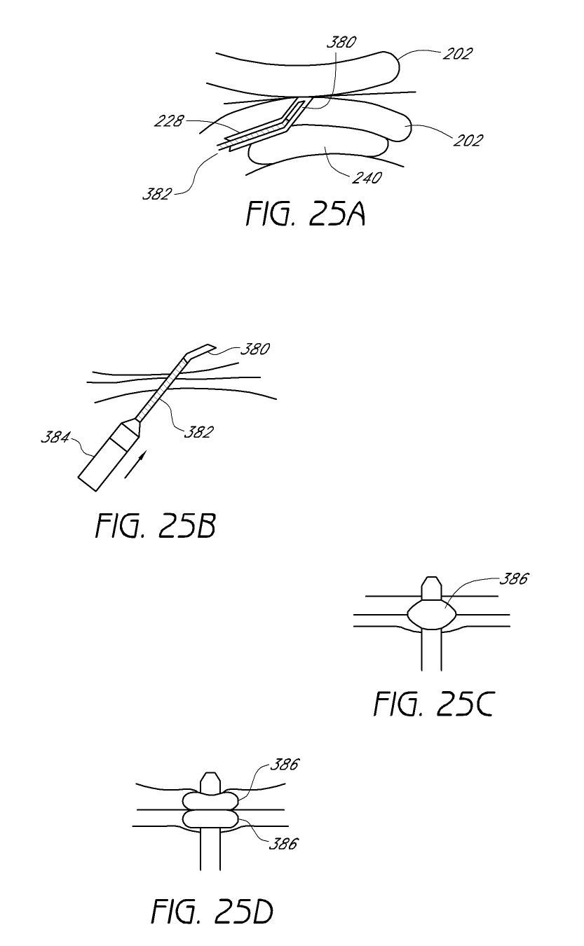

In other embodiments, illustrated in FIGS. 25A-25D, in order to anastomose the jejunum to the stomach without sandwiching the omentum, for example in patients with obesity, where the omentum has been demonstrated to be thick (frequently in obese males), a needle 380/guide wire 382 assembly can be inserted through a side-directed lumen 228 and be delivered across the two walls and the catheter 202 can be removed. A specialized balloon catheter 384 can be advanced and placed over the guide wire 382. The guide wire 382 can be removed and the balloon 384 inflated to dilate the omentum. Insufflated, the balloon shape can be like a sphere, an ovoid, a doughnut or a red blood cell, or a figure-8 profile with a waist, or two adjacent balloons, or the like, helping to do a controlled blunt dissection (or circular tear) into the omentum between the two apposed walls of stomach and jejunum. In some embodiments, a two-balloon configuration, see FIG. 25D, can form a waist to capture omentum. Once the maximum size is reached (approximately the size of a half dollar), and the stomach and the jejunum are adequately apposed, the welding or other anastomosing can begin. In this case, or in the case of the anastomosis, tissue glue can be injected through the injection needle 380 into the space between the tissues to further seal the anastomosis on the outside. The omentum serves the immune defense in the abdominal cavity and is used in surgery to cover and protect delicate anastomoses. Thus, the procedure described above provides a natural protective element being added to the anastomosis.

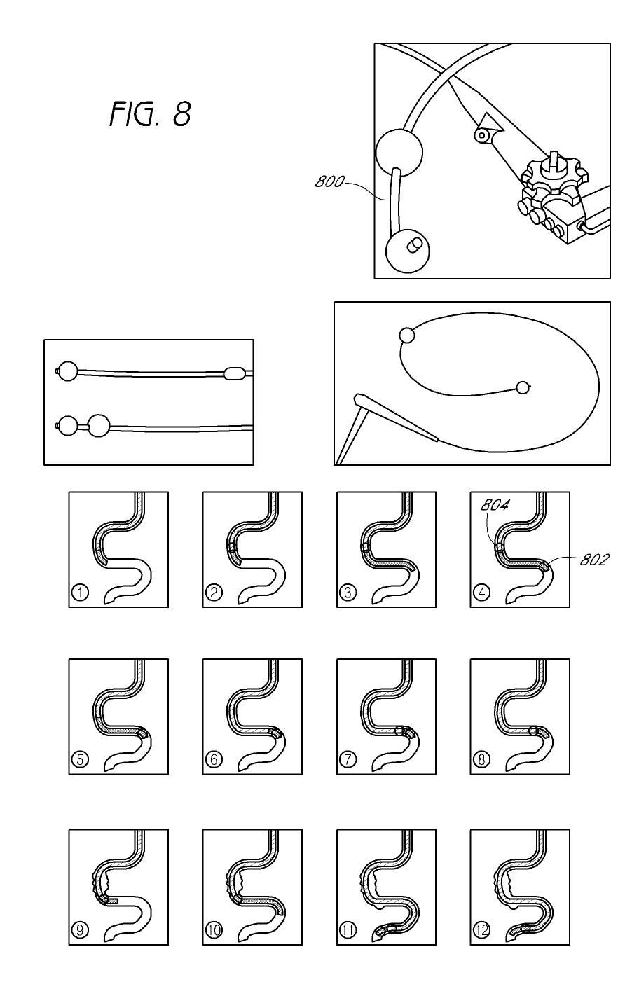

In other embodiments, illustrated in FIG. 8, a two-balloon technique, applying a two-balloon device 800, can be used to bring the second target 118 close to the first target 116. A two-balloon surgical technique uses a first balloon 802 to anchor the intestine, a co-axial sliding second balloon 804 catheter to anchor a second distal location. Egress of the second balloon toward the first balloon foreshortens the intestine loop.

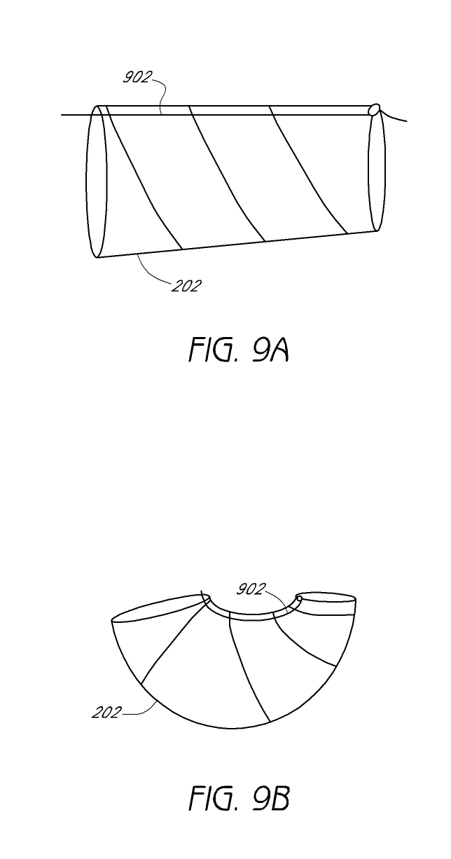

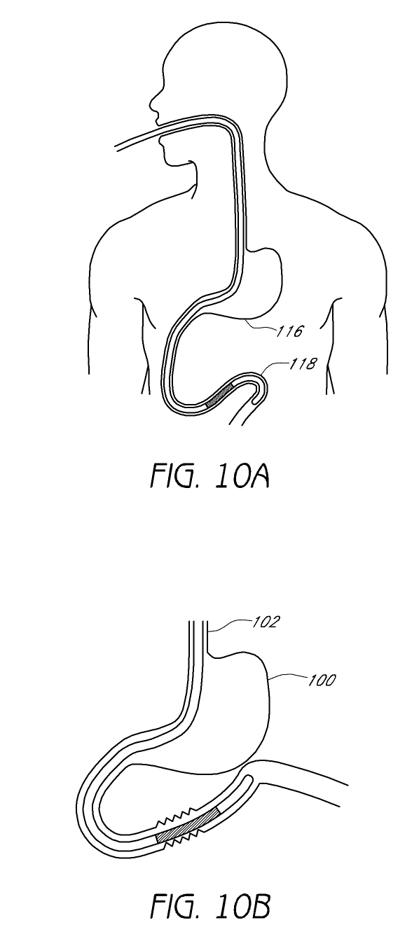

In other embodiments, a standard shape can be predetermined for the catheter 200, such that the catheter 200 can be deployed in a first flexible state, and then activated to become the predetermined shape. Simple tension lines 902, illustrated in FIG. 9, within a spring shaft can be activated to force the predetermined shape. Thus, the catheter 200 can be made to bend, with the tip coming to rest alongside its own shaft. With the tip in one lumen and the shaft in another, this action would bring the two sites together to be anastomosed. In one embodiment, the predetermined shape catheter 200 can be combined with the two-balloon device 800 technique (see FIG. 10) such that the distance to be traversed is reduced, a rigid proximal length of catheter can be used for leverage, and the arc traversed by the deploying catheter 200 can be minimized.

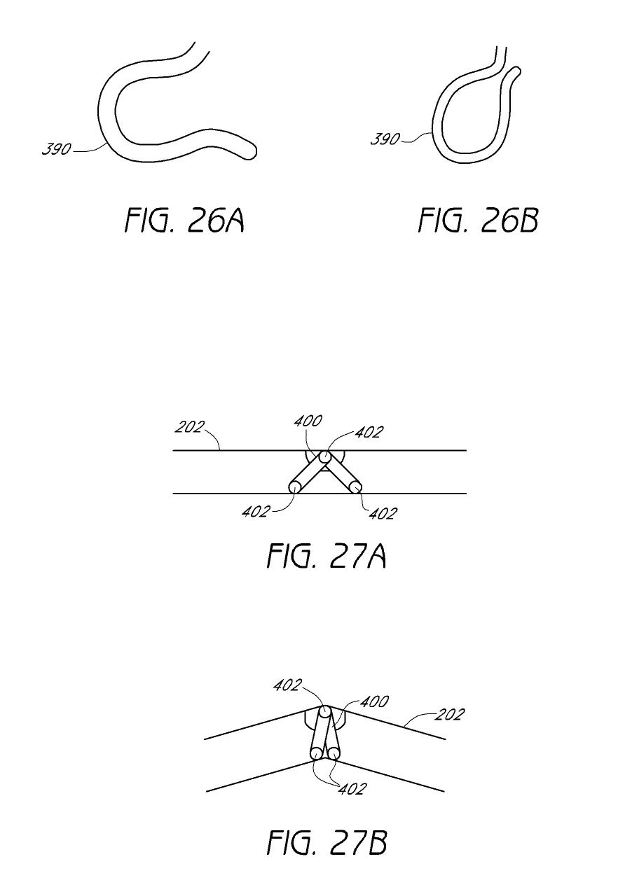

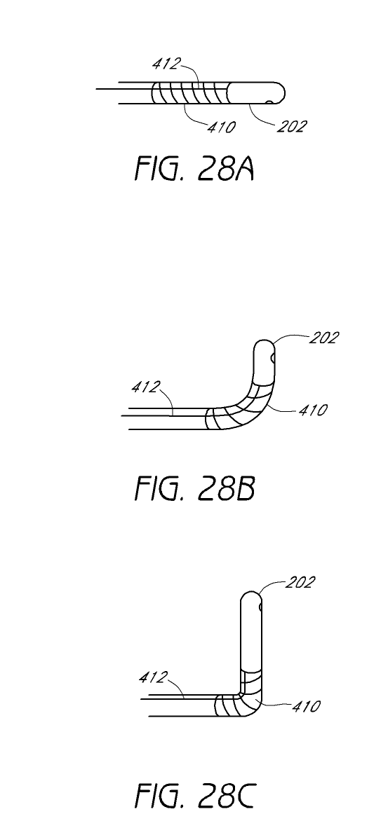

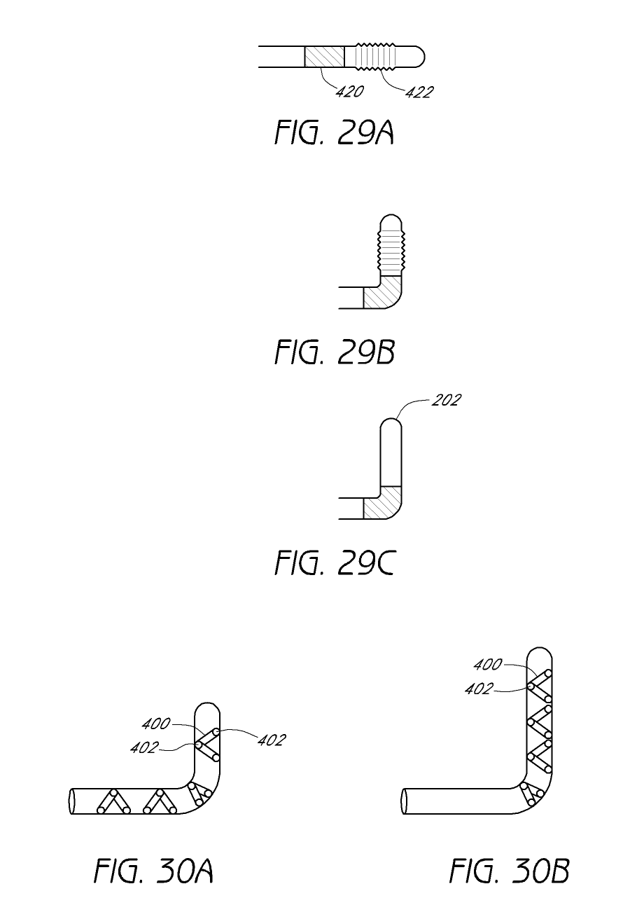

In other embodiments, illustrated in FIGS. 26A-B, hydraulic or pneumatic devices can be used to inflate the catheter, rigidizing the catheter 390 into its predetermined shape. FIG. 26B shows the catheter 390 inflated. The shape can be a balloon with walls containing flexible, high tensile strength, low denier threads, such as a braid of Kevlar or UHDPE, or the like. High tension threads laid along stress lines are highly flexible when uninflated. Inflation of the catheter 390 causes the shorter thread to form an inside radius of a curve, or bend, in the catheter 390 shape. In other embodiments, illustrated in FIGS. 27A-B, electronically activated actuators 400 on a series of joints 402 can be individually operated to allow a catheter 202 to move in a predetermined sequence. These joints 402 can have a single degree of freedom, since only one turn is required. In still other embodiments, illustrated in FIGS. 28A-C, a sheath 410 can be slidably placed externally to the catheter 202 and forced into a bent position to form an elbow in place. The sheath 410 can include a tension line 412 that can be tensioned to form the bent position. The catheter 202 can then pass through this directing elbow of the sheath 410. In other embodiments, illustrated in FIGS. 29A-C, a shaft with a single elbow 420 can be positioned with the elbow relaxed, then the elbow can be bent and an accordion, or pleats 422, on the other side of the elbow can be deployed (FIG. 29B). In all these ways, a catheter can be made to bend, with the tip coming to rest alongside its own shaft. With the tip in one lumen and the shaft in another, this action would bring the two sites together to be anastomosed. In particular, in the case of the multiple articulated catheter with electronic actuators, the tip can be bent around a very small radius (see FIG. 30A-B), and then pushed forward. The radius can remain in position relative to the tissue, while progressing proximally relative to the catheter. As the catheter 202 is pushed forward the actuators 400 are operate independently to maintain a bend at an anatomical location.

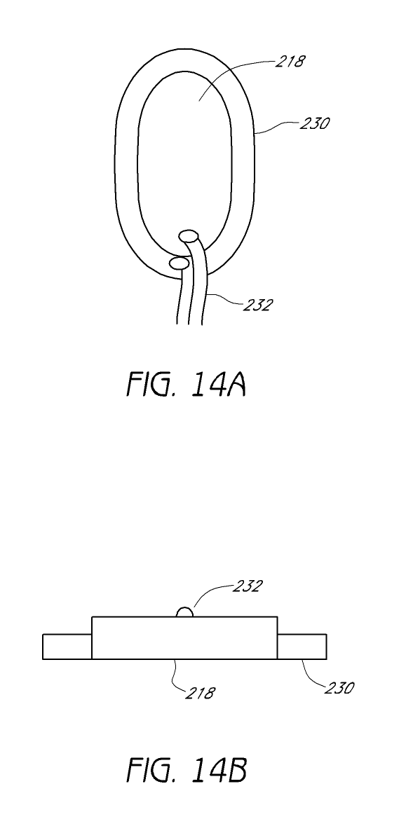

The joining members, or electrode 230, are the welding members that are activated at the target site to perform the tissue welding. In the illustrated embodiment of FIGS. 14A and 14B, the electrodes 230 can take any desired shape suitable for deployment and coupling to the electromagnets, or magnets 218. The electrode 230 can be flat with an exterior dimension defining the weld pattern and the width of the weld. The shape can be optimized for the tissue welding process, and to control the flow of chyme (valve) and mitigate the risk of contamination and collateral risk during the surgical procedure. In one embodiment, the electrode 230 can include end radii, e.g. an oval or equivalent rounded shape, to prevent tissue tearing during the deployment and anastomosis procedure. In one embodiment, the electrode can be a semi-rigid structure, or alternatively, be flexible to allow for adjustment of the shape during the approximating operations.

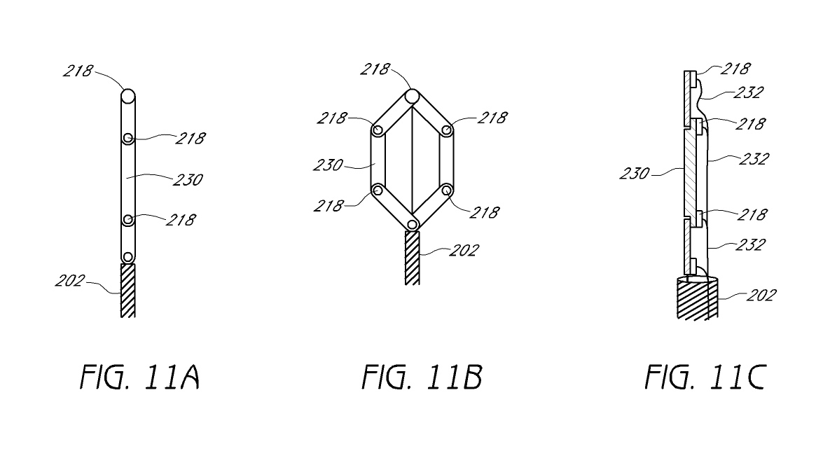

In another embodiment, illustrated in FIGS. 11A-11C, the weld member electrodes 230 can be flat with an exterior dimension defining the weld pattern and the width of the weld, as described above. The exterior dimension can be a pair of jointed bands 1106 joined by a deployment driveshaft 1102. The joints 1104 of the jointed band 1106 can include electromagnets 218 used for the approximation process of aligning the first and second targets 116, 118. Application of tension on the driveshaft 1102 can cause the jointed bands, or arms 1106 to flare outward and form an oval-like shape. The joints 1104 electromagnets can be activated to force the opposing elements of the two facing electrodes 230 to lay flat against each other for welding of the tissue.

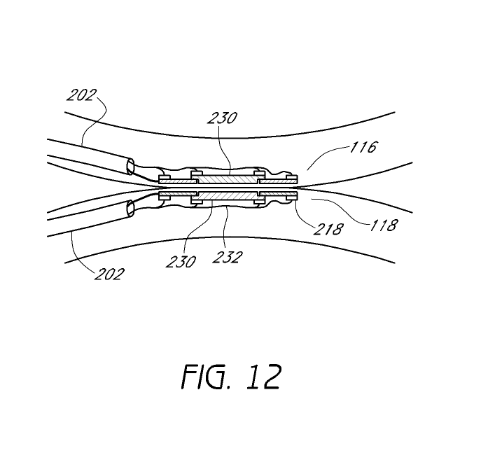

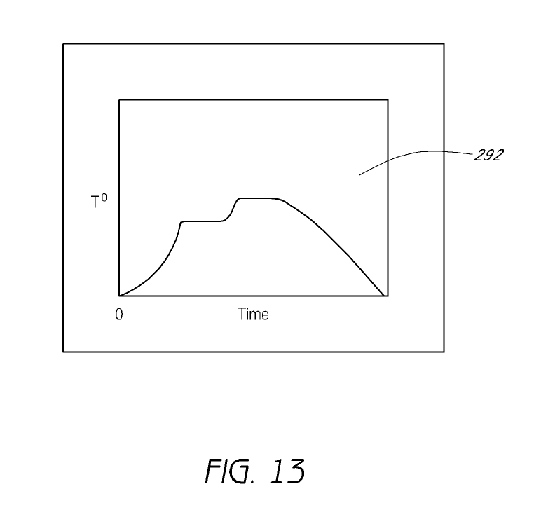

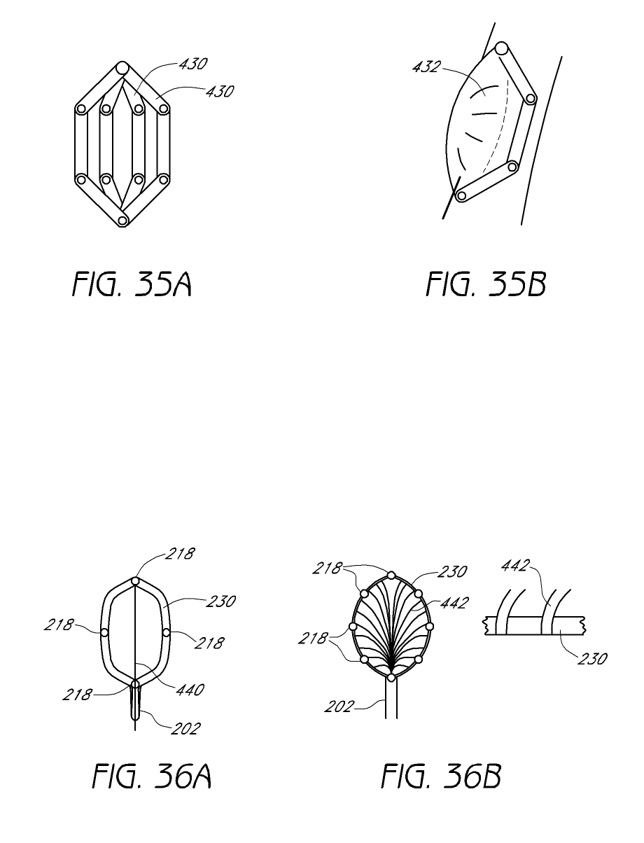

In the illustrated embodiment of FIG. 12, the two electrodes 230 are deployed and aligned opposite the tissue walls to be welded. Once deployed, the welding arms can be activated with RF energy to weld the two target sites 116, 118 together along the perimeter of the electrodes 230. In one embodiment, the tissue welding can be performed without injecting tissue adhesives 432 into the captive site, or newly formed serosal cavity, for anastomosis. In other embodiments, a tissue adhesive can be injected into the captive site for anastomosis (see FIG. 35B). In other embodiments, the welding arms of electrode 230 can include multiple electrode leaflets 430 establishing more than one circumferential weld pattern (see FIG. 35A). Tissue welding parameters can include such variables as time, temperature, and pressure to obtain an adequate welding of adjacent tissue. The required compression pressure can be obtained via the electromagnets. Time, frequency, and power for welding can be controlled by a standard RF generator via the controller 216, or an alternate external control mechanism (see FIG. 13) to the parameters of, for one embodiment, a control profile 292. In other embodiments, illustrated in FIGS. 36A-36B, alternative energy sources for welding include DC electricity, light, microwave, and ultrasound. Additionally, alternative deploying arms can be fiberoptic arrays, piezo arrays, or microwave antennae (see FIGS. 36A-B). The deploying arm can include round wire antenna for microwave or RF energy delivery. The electrodes can include a tension element 440 that can be tensioned to deploy the antenna. In some embodiments, the electrode can include magnets at hinge points. In some embodiments, the electrode can include a fiberoptic bundle coupled, or potted, in the arm elements of the electrode at various positions about the arms of the electrode and which are flexible and expandable to the deployed position of the arms. The fiberoptics can be potted into the arms of the electrode 230

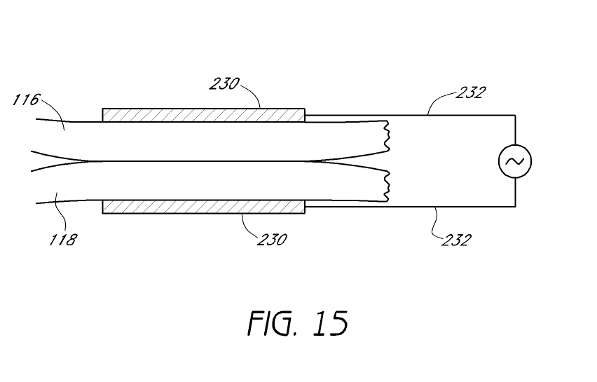

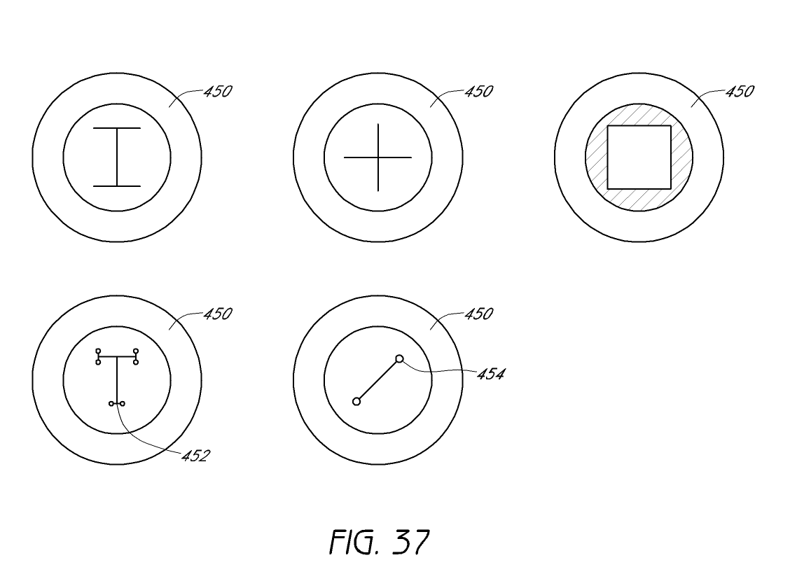

In one embodiment, the opposing deploying arms 230 can be part of a single circuit, passing current through the tissue for welding (see FIG. 15). Once the anastomosis is completed the tissue will be opened by cutting the tissue internal to the tissue weld regions 450. In one embodiment, the tissue is cut using ablative heating. The electromagnet approximators can have a second band surrounding their outer diameter. This second band can be heated with a DC current to a temperature that will char a ring of tissue. Once charring is complete, the electromagnets 230 will be withdrawn together in the oral direction to ensure that ablation has completely freed a tissue disc. The electromagnets 230 can be disengaged and retracted by their power members 232, or electrical connections, after the opening is complete. Alternative anastomotic opening shapes can be implemented, e.g. a slit, a cross, a T-shaped opening, a rectilinear opening, or the like (see FIG. 37). The sharp-angled end points of such opening shapes can be strengthened by application of a suture, a staple 452, an additional larger radius such as a hole-punch diameter 454, to mitigate a stress riser geometry, provide a stress relief, and prevent tearing of the tissue (see FIG. 37).

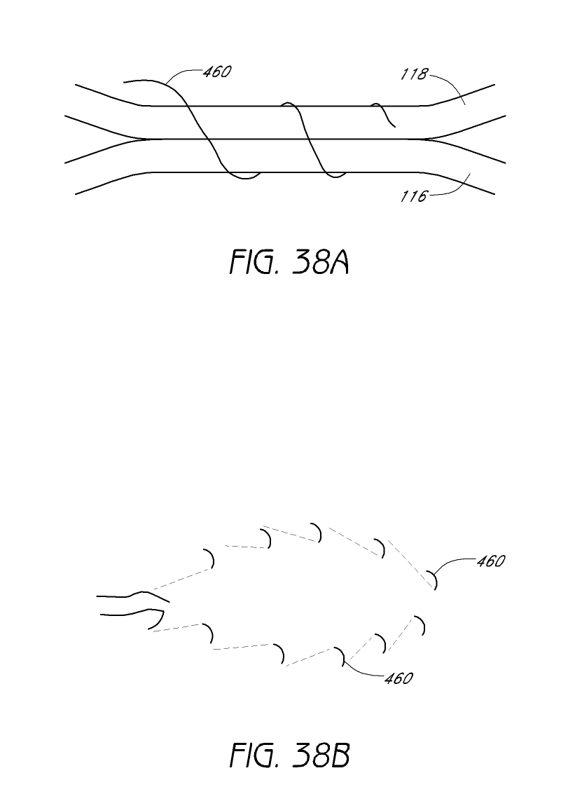

In other embodiments, illustrated in FIGS. 38A-38B, rather than welding the two tissues together, two corkscrew needles 460 can be screwed into the wall to hold the two tissues together, and the tissue between the needle can be opened. The needles can be metal such as stainless steel, or can be a rigid polymer such as PEEK, or can be a rigid bioresorbable polymer such as poly (lactic acid) stereocopolymer. Alternatively, the arms can be magnets and can remain in place.

In still other embodiment, illustrated in FIG. 39, the arms can be magnets 470 and a tightly sealed ring can be formed. An adhesive 432 can be injected into the external serosal pocket, filling the pocket. Subsequently the opening can be formed as before, and the magnets retracted. Rather than glue, an irritant can be injected into the serosal pocket, such as hydrochloric acid or other FDA-cleared irritants. Fibrin release will form a bond between the two tissue surfaces, and a collagen scar will bind the two surfaces. A perforation can be formed with a second procedure when scarring is complete. Alternatively, stents 352 can be deployed with a specific anastomosis shape, as described above. These stents can include an obstructive element at one end.

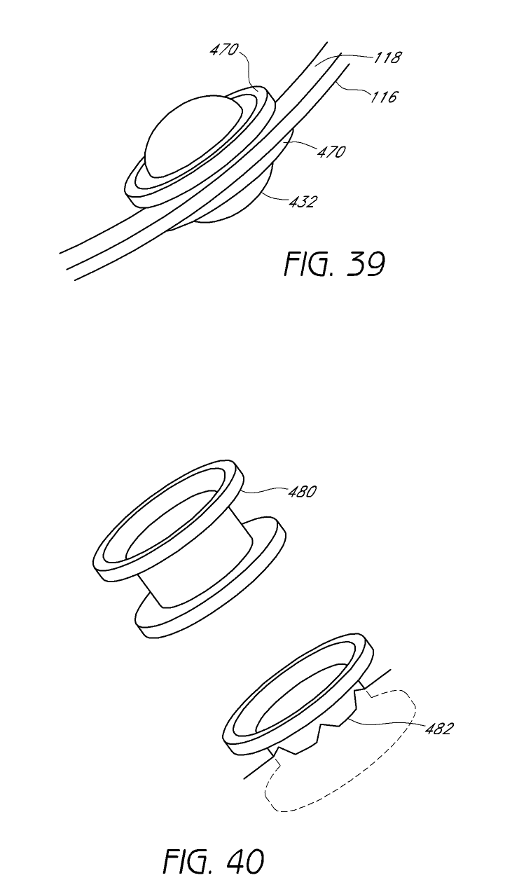

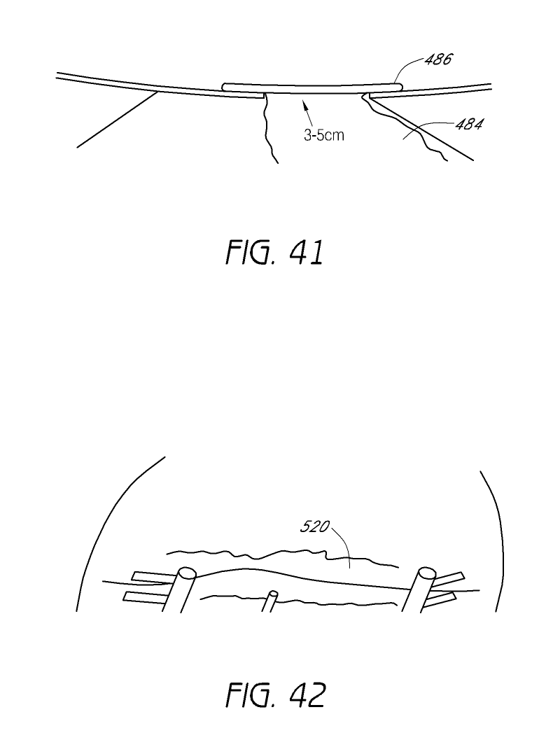

In some embodiments, illustrated in FIG. 40, an alternative to burning to establish the opening is provided by a simple cutting tool that can be deployed with visualization. Alternatively, an implanted form, implant 480, can produce the desired opening shape. One of the other openings, such as a slit 482 or cross or T can be formed and the implant can be forced across the opening, such as an x-shaped slit. The implant 480 would provide a permanent, fixed, optimized opening shape that includes a permanent flanged orifice. The implant 480 can be a metal or polymer acceptable for long term implantation. It can be in the form of a grommet. To provide an inner seal for the anastomosis and to prevent leakage or even promote healing, a flexible (possibly funnel-shaped) sheath 484 with an oral stabilizer ring 486, or flanged orifice, previously inserted in a collapsed form, can be placed into the anastomosis or stoma (see FIG. 41). The flexible sheath can cover the inside of the anastomosis to about 3-5 cm. The advantage of such a stent is to provide a secure channel to cross between two lumina.

Advantageously, performing the anastomoses via the described method allows the tissue to be joined prior to the opening in the tissue being created. Joining before cutting prevents spillage of the contents of the digestive system, or tract, into the peritoneal cavity. The described method, therefore, mitigates the risk of persisting leaks of digestive tract spillage, which can be life-threatening. The described apparatus and method of anastomosis also advantageously eliminates the need to dissect the omentum to allow bowel or stomach approximation for the selected anastomosis sites. Additionally, the magnet 218 and the electrode 230 can be designed to define the actual anastomotic orifice for optimal performance, including flow performance and prevention of obstruction.

In one embodiment of the illustrated method, the second location 130 is also anastomosed between the third location 122 and the fourth location 124. Using any of the above described apparatuses and/or methods, the third location 122 and the fourth location 124 are identified and marked with magnets 218, at least one magnet 218 at each of the two locations. The magnets 218 are then manipulated adjacent one another, as described above, and joined together via tissue welding as described above. The magnets 218 at the third location 122 and the fourth location 124 can be deployed from the catheter 200. In some embodiments, the magnets can be deployed from a second, separate, insertion device. In some embodiments, one of the magnets 218 at either third location 122 or fourth location 124 can be deployed from the catheter 200 and the other magnet 218 can be deployed from a second, separate, insertion device. The second location 130 can be manipulated and joined after the first location 120 is anastomosed and the opening cut, thereby allowing access through the opening to either or both of the third location 122 and the fourth location 124. After the second location 130 is joined, the area of tissue internal to the region of tissue welding can form an opening, e.g. by cutting the tissue. In some embodiments, the tissue can remain without an opening. In one embodiment, one or more deployment catheters can be inserted through the opening formed at the first location 116 such that the catheter, the duodenum 108, and the jejunum 110 can be bypassed.

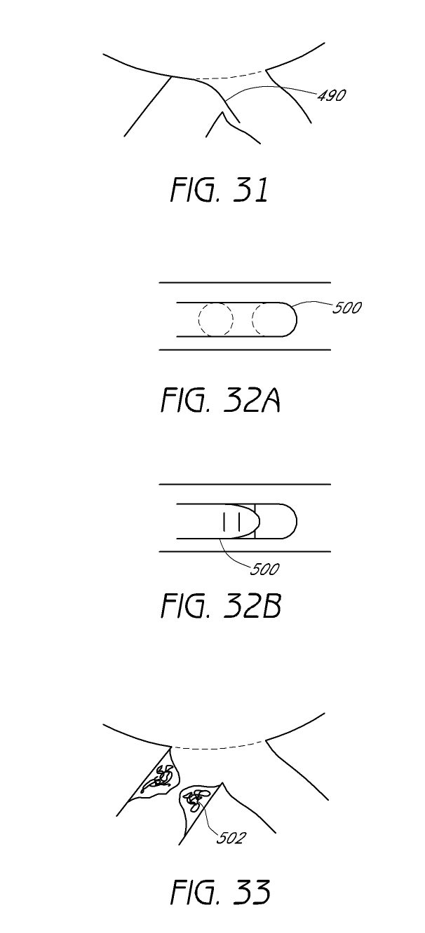

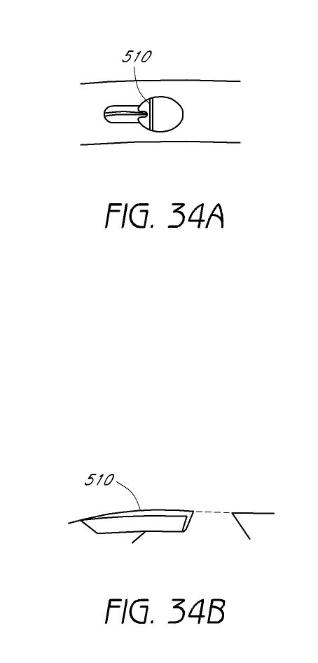

In other embodiments, illustrated in FIGS. 31-34, creation of a valvular mechanism through specific anastomotic features will prevent “marginal” or stoma ulceration by preventing jejuno-gastric reflux. This can be achieved by inverting the stomach wall 490 into the jejunum, preferably only the alimentary loop, creating a one-way valve mechanism 490, or by creating uneven stomata at the anastomotic site with a large alimentary or efferent loop stoma and a narrow afferent loop stoma, by welding or suturing/stapling the afferent loop into a flap that occludes the afferent stoma. FIG. 32A illustrates an incision for the tissue flap 500 in an internal top view. FIG. 32B illustrates a top view of the tissue flap 500 collapsing to partially obscure the efferent loop. A specific and asymmetric incision of the welded or approximated anastomosed tissue can result in an opening favoring the efferent loop and keeping the afferent loop largely blocked. Though complete closure can be beneficial, a small orifice can be useful to provide drainage. Injection of suture material 502 or other FDA-cleared biocompatible materials like steel or nitinol beads, silicone and other polymers, or the like, into the submucosal space can also narrow the afferent loop stoma. FIG. 32C illustrates a side view with suture 502 injected submucosal to obstruct the efferent loop. FIGS. 34A-B illustrates a tissue pleat 510 formed wherein the tissue pleat encroaches on efferent stoma.

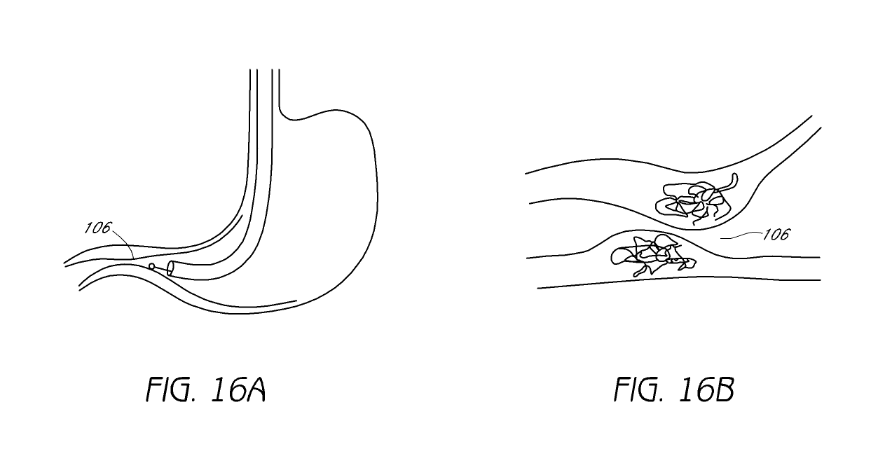

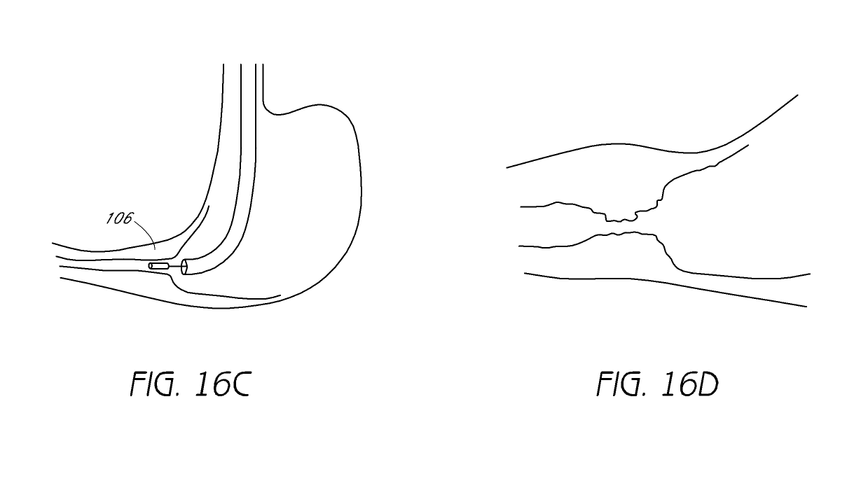

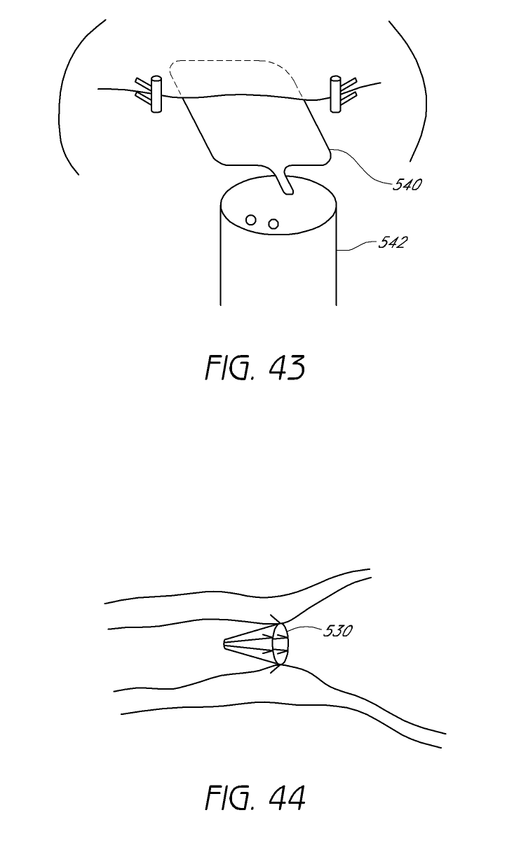

In addition to the anastomosis and the opening, two sites must be occluded, as illustrated in FIGS. 16A-16B, in order to prevent or significantly reduce flow to ensure the proper directionality of chyme travel. Complete occlusion is not necessary, but variation in performance of the type 2 diabetes effect may not be predictable if the occlusion is not complete. Thus, complete occlusion and therefore complete segregation of the secretion arm from chyme or the alimentary arm is preferred. The obstruction to establish the occlusion may be formed by scar tissue created by use of a heating element and visualization via a camera. The pylorus is thick-walled and can be the preferred target for the obstructive damage. In other embodiments, a heated blade can be used to weld the tissues together (see FIG. 43).

In other embodiments, illustrated in FIGS. 16A-16D and 42-44, a volume of non-resorbing biocompatible material such as suture can be injected into the wall of the lumen to form an obstructive bulge. Other materials such as stainless steel or nitinol beads, silicone, or other well-characterized polymers can be injected. Alternatively, a sclerosing agent such as alcohol can be injected into the wall to cause retraction and scarring. Alternatively, a ring of mucosa 520 can be abraded with an abrasive tool, and the ring can be forced closed to heal into an obstruction. The ring can be forced closed with staples, suture, pins, clips, or rivets. Laser energy can weld the abraded tissues 520 together (see FIG. 42). A flat heated blade 540 can be withdrawn while heating the tissue with the use of an endoscope 542. Alternatively an obstructive implant 530, such as a ball, sponge, or an umbrella with anchor hooks (see FIG. 44), can be placed into the lumen, or deployed at the pylorus. These can be placed after the mucosa has been abraded, to cause healing adhesion. The obstruction 530 can have anchor hooks or protrusions to prevent dislodgement. Alternatively, the obstructive implant 530 can be a combination of a physical implant such as a electroconducting ball or mesh that is being brought into position, energy is turned on and as the mucosa is being partially or totally ablated, the lumen shrinks around the implant, the electrode is being decoupled from the implant and withdrawn, while the implant is left behind.

The occlusion should be positioned such that a long column of immobile chyme will not form, as this may stagnate. In addition, reverse flow through the anisoperistaltic loop may not be likely, due to its natural motility and peristaltic motion. However, it is important that bile not leak into the anastomotic site, as it will erode tissues. The length of the anisoperistaltic loop can prevent bile-induced erosion of the anastomotic site. Chyme that travels in the antiperistaltic direction in this loop will not cause any reduction in the type 2 diabetes cure effect.

In light of the disclosure herein, in some embodiments, one can varying lengths of bowel at which the anastomosis is placed intraluminally and extraluminally, which allows for dialing in different levels of control of different types of severity of diabetes and/or obesity. Accordingly, some arrangements, comprise selecting different lengths of the jejunal portions between anastomses ⅓ and 2/4, resulting in different length of bypassed upper intestines (see FIG. 1). In addition, the length and materials (bioabsorbable/degradable and permanent/non-absorbable) for the lumen-crossing sleeves that a) protect the anastomoses, can be varied to allow for fine-tuning of absorption control.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. For example, the embodiments disclosed above can be used with gastric bypass procedures targeting other locations of the digestive system for anastomosis. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. A method of gastric bypass surgery comprising:
advancing a first device to a first target site within a digestive tract of a patient;
manipulating the first device inside and/or outside the patient to move the first target site approximate to a second target site within the digestive tract;
joining the first and second target sites together to form a junction with a periphery; and
forming an opening within the periphery of the junction.

2. The method of claim 1, wherein the step of manipulating the first device comprises activating a magnet positioned at the first target site.

3. The method of claim 1, wherein the step of manipulating the first device comprises articulating a distal end of the first device.

4. The method of claim 1, wherein the step of joining the first and second target sites together comprises welding tissue together.

5. The method of claim 1, further comprising advancing a second device or the first device to a third target site and manipulating the second device or first device outside the patient to move the third target site approximate to a fourth target site within the digestive tract.

6. The method of claim 5, further comprising joining the third and fourth target sites together to form a junction with a periphery.

7. A method of treating diabetes comprising:
inserting an elongate member orally through the digestive tract, the elongate member having an internal volume, a proximal end, and a distal end, the internal volume extending from the proximal end to the distal end;
locating a first biological matter location and a second biological matter location with the distal end of the elongate member;
deploying a first coupler at the first biological matter location and a second coupler at the second biological matter location from the distal end of the elongate member, the first coupler and the second coupler deploying from the internal volume of the elongate member and maintaining a coupling to the internal volume;
attaching a first protrusion coupled to the first coupler to the first biological matter location and a second protrusion coupled to the second coupler to the second biological matter location;
manipulating the second coupler adjacent the first coupler by directionally maneuvering an external coupler about the second coupler;
aligning the first coupler with the second coupler;
joining the first biological matter location to the second biological matter location by activating a first joining member coupled to the first coupler and a second joining member coupled to the second coupler;
opening the joined portion of the first biological matter location and the second biological matter location to provide for flow of bodily fluid;
disengaging and retracting the couplers from the first and second biological matter locations; and
removing the elongate member from the digestive tract.

8. The method of claim 7, further comprising locating a third biological matter location and a fourth biological matter location, deploying a third coupler and a fourth coupler, manipulating the third coupler outside the patient approximate the fourth coupler, joining the third biological matter to the fourth biological matter, and forming an opening of the third biological matter to the fourth biological matter.

9. A medical treatment device comprising:
an elongate member having an internal volume, a proximal end, and a distal end, the internal volume extending from the proximal end to the distal end;
a first coupler and a second coupler, the first coupler and the second coupler coupled to the internal volume of the elongate member;
a first joining member and a second joining member, the first joining member coupled to the first coupler and the second joining member coupled to the second coupler;
wherein the first joining member is configured to attach to a first biological matter location, and the second joining member is configured to attach to a second biological matter location, the second location being distal to the first location, and wherein the second coupler is configured for manipulation to align relative the first coupler such that the second biological matter location relocates adjacent the first biological matter location, the first joining member and the second joining member configured to join the first biological matter location to the second biological matter location.

10. The medical treatment device of claim 9, wherein a first indicator is coupled to the first coupler and a second indicator is coupled to the second coupler, the first indicator and the second indicator configured to indicate an alignment between the first coupler and the second coupler.

11. The medical treatment device of claim 9, wherein the first joining member and the second joining member are electrodes, and the first joining member and the second joining member can couple together biological matter when an external power source is provided to the first and second joining members.

12. The medical treatment device of claim 9, wherein the first joining member and the second joining member are oval-shaped, having an aperture extending from a first face to a second face.

13. The medical treatment device of claim 12, wherein the aperture shape is optimized for bodily fluid flow.

14. The medical treatment device of claim 9, wherein the first coupler and the second coupler comprise electromagnets.

15. The medical treatment device of claim 9, wherein the manipulation of the second biological matter location relative the first biological matter location is provided by a reversible electromagnetic power source to the second coupler and the first coupler.

16. A method of treating obesity comprising:
inserting an elongate member orally through the digestive tract, the elongate member having an internal volume, a proximal end, and a distal end, the internal volume extending from the proximal end to the distal end;
locating a first biological matter location and a second biological matter location with the distal end of the elongate member;
deploying a first coupler at the first biological matter location and a second coupler at the second biological matter location from the distal end of the elongate member, the first coupler and the second coupler deploying from the internal volume of the elongate member and maintaining a coupling to the internal volume;
attaching a first protrusion coupled to the first coupler to the first biological matter location and a second protrusion coupled to the second coupler to the second biological matter location;
manipulating the second coupler adjacent the first coupler by directionally maneuvering an external coupler about the second coupler;
aligning the first coupler with the second coupler;
joining the first biological matter location to the second biological matter location by activating a first joining member coupled to the first coupler and a second joining member coupled to the second coupler;
opening the joined portion of the first biological matter location and the second biological matter location to provide for flow of bodily fluid;
disengaging and retracting the couplers from the first and second biological matter locations; and
removing the elongate member from the digestive tract.

17. The method of claim 16, further comprising selecting different lengths of bypassed upper instenstines.
